(19) United States
(12) Patent Application Publication
(10) Pub. No.: US 2019/0105187 A1
(43) Pub. Date: Apr. 11, 2019

(54) FOOT SUPPORTING GARMENTS AND METHODS

(71) Applicant: IntelliSkin USA, LLC, Woodbury, NJ (US)

(72) Inventor: Timothy W. Brown, Newport Beach, CA (US)

(21) Appl. No.: 16/212,306

(22) Filed: Dec. 6, 2018

Related U.S. Application Data
(63) Continuation of application No. PCT/US2017/036422, filed on Jun. 7, 2017.
(60) Provisional application No. 62/346,986, filed on Jun. 7, 2016.
(60) Provisional application No. 62/471,906, filed on Mar. 15, 2017.

(51) Int. Cl. A61F 5/01; A41B 11/08

(52) U.S. Cl. CPC A61F 5/0111; A41B 11/08; A41B 2400/82; A41B 2300/22; A41B 2400/32

(57) ABSTRACT

A garment for reducing pain associated with pes planus includes an ankle band having an anterior portion and a posterior portion; and a foot portion that includes a first band wrapping around the foot portion. The first band has a superior portion and an inferior portion. The garment also includes a plurality of lift bands extending from the anterior portion of the ankle band to at least the superior portion of the first band so as to provide a pull on the superior portion of the first band toward the anterior portion of the ankle band.

### RELATED APPLICATIONS

This application is a continuation application of International Patent Application Serial No. PCT/US2017/036422, filed Jun. 7, 2017, which claims priority to U.S. Provisional Patent Application Ser. No. 62/346,986, filed Jun. 7, 2016 and U.S. Provisional Patent Application Ser. No. 62/471,906, filed Mar. 15, 2017. All of these applications are incorporated by reference herein in their entireties.

### TECHNICAL FIELD

This application relates generally to garments configured to be worn on a user's foot (e.g., socks), including but not limited to garments that support proper movement, function, alignment, and posture of the foot.

### BACKGROUND

Shoes and/or sedentary lifestyles reduce the mobility, coordination, stability and strength of muscles that support the proper movement, function, alignment, and posture of a foot.

A foot is an incredible feat of engineering. It includes 26 bones, 33 joints, and well over 100 ligaments, tendons, and muscles. About a quarter of all bones in a person can be found in the person's feet. Just like the rest of the muscles in a human body, foot muscles function better with regular exercise that helps them remain flexible and strong so they can absorb the shock of impact and transfer power up the leg when walking, running and performing other activities of daily living.

One of the most common problems with the foot is weak arches or flat feet (e.g., pes planus). When driving down a bumpy road with a car with great shock absorbers, good shock absorption provides a smooth and comfortable ride and serves to prevent damage to other parts of the car. In comparison, like driving a car down the same bumpy road with no shock absorbers on the car, flat feet cannot absorb shocks well during walking, running, and other daily activities. Having poor arches increases the chance of causing other serious foot problems such as plantar fasciitis, Morton's neuroma, ankle sprains, posterior tibialis tendonopathy, and more.

### SUMMARY

Thus, there is a need for a device or a garment that can better support proper movement, function, alignment, and posture of a foot. Having strong healthy feet provides a foundation for a healthy body by allowing a wearer to improve how the wearer moves.

In accordance with some embodiments, a garment for supporting a wearer's foot includes a first band configured to wrap around at least a portion of navicular, cuboid, and cuneiform bones of the wearer's foot when the garment is worn on the wearer's foot; and a second band intersecting with the first band and configured to wrap around a heel of the wearer's foot when the garment is worn on the wearer's foot. The second band is configured to support medial and lateral surfaces and at least a portion of a plantar surface of the heel.

In some embodiments, the second band is configured to expose at least a portion of the plantar surface of the heel when the garment is worn on the wearer's foot.

In some embodiments, the second band crisscrosses over at least a portion of navicular, cuboid, and cuneiform bones of the wearer's foot when the garment is worn on the wearer's foot.

In some embodiments, the second band is separate and distinct from the first band.

In some embodiments, the second band is configured to pull the heel of the wearer's foot from an inferior surface of the wearer's foot toward a superior portion of the wearer's foot, and from a posterior portion of the wearer's foot toward an anterior portion of the wearer's foot when the garment is worn on the wearer's foot.

In some embodiments, the garment further includes a third band configured to wrap around at least a portion of metatarsals of the wearer's foot when the garment is worn on the wearer's foot.

In some embodiments, the second band extends from a medial portion of the third band to a lateral portion of the third band while wrapping around the heel of the wearer's foot when the garment is worn on the wearer's foot.

In some embodiments, the garment further includes a plurality of bands configured to pull the third band toward an ankle of a wearer when the garment is worn on the wearer's foot.

In some embodiments, the garment further includes an ankle band configured to wrap around the ankle of the wearer. The plurality of bands extends from the third band to the ankle band.

In some embodiments, the ankle band is separate and distinct from the second band.

In accordance with some embodiments, a garment includes an ankle band that includes an anterior portion and a posterior portion; and a foot portion that includes a first band wrapping around the foot portion. The first band has a superior portion and an inferior portion. The garment also includes a plurality of lift bands extending from the anterior portion of the ankle band to at least the superior portion of the first band so as to provide a pull on the superior portion of the first band toward the anterior portion of the ankle band.

In some embodiments, the foot portion includes an anterior portion and a posterior portion. The first band is located at least partially within the posterior portion of the foot portion.

In some embodiments, the garment further includes a second band that wraps around the foot portion and is located at least partially within the anterior portion of the foot portion. The second band has a superior portion and an inferior portion, and one or more lift bands of the plurality of lift bands extend from the anterior portion of the ankle band to at least the superior portion of the second band so as to provide a pull on the superior portion of the second band toward the anterior portion of the ankle band.

In some embodiments, the foot portion includes an anterior portion and a posterior portion. The first band is located at least partially within the anterior portion of the foot portion.

In some embodiments, the first band has a medial portion and a lateral portion. The first band is positioned to contact a skin of a wearer when the garment is worn on a foot of the wearer. The garment also includes a friction layer located on the medial portion of the first band to pull up a navicular bone of the wearer when the garment is worn on the foot of the wearer.

In some embodiments, the first band has a medial portion and a lateral portion. The first band has a first thickness at the medial portion and a second thickness, that is less than the first thickness, at the lateral portion.

In some embodiments, the garment further includes a heel portion; and a heel band intersecting with the first band and wrapping around at least a portion of the heel portion so as to pull the heel portion toward the foot portion.

In some embodiments, the plurality of lift bands includes at least one lift band extending from the anterior portion of the ankle band to at least the superior portion of the first band at least partially over a middle metatarsal bone.

In some embodiments, the first band has a medial portion and a lateral portion. At least two lift bands of the plurality of lift bands extend from the anterior portion of the ankle band to the medial portion of the first band. At least one lift band of the plurality of lift bands extends from the anterior portion of the ankle band to the lateral portion of the first band.

In some embodiments, the garment is a sock.

In accordance with some embodiments, a garment includes a foot portion that includes a first band wrapping around the foot portion. The first band has a medial portion and a lateral portion. The first band is positioned to contact a skin of a wearer when the garment is worn on a foot of the wearer. The garment also includes a friction layer located on the medial portion of the first band to pull up a navicular bone of the wearer when the garment is worn on the foot of the wearer.

In accordance with some embodiments, a method for reducing pain associated with pes planus includes wearing, on a foot, a garment that includes an ankle band that includes an anterior portion and a posterior portion; and a foot portion that includes a first band wrapping around the foot portion. The first band has a superior portion and an inferior portion. The garment also includes a plurality of lift bands extending from the anterior portion of the ankle band to at least the superior portion of the first band. The method also includes pulling the superior portion of the first band toward the anterior portion of the ankle band and lifting a navicular bone on a medial portion of the foot.

### BRIEF DESCRIPTION OF THE DRAWINGS

The garments, together with additional features and advantages thereof, may be best understood by reference to the following description taken in conjunction with the accompanying illustrative drawings. In these accompanying drawings, like reference numerals designate like parts throughout the figures.

FIG. 1 is a schematic diagram illustrating a plan view of foot bones.

FIG. 2 is an inner side elevation view of a garment in accordance with some embodiments.

FIG. 3 is an inner side elevation view of a garment in accordance with some embodiments.

FIG. 4 is an outer side elevation view of the garment shown in FIG. 3.

FIG. 5 is a top view of the garment shown in FIG. 3.

FIG. 6 is a bottom view of the garment shown in FIG. 3.

FIG. 7 is an inner side elevation view of a garment in accordance with some embodiments.

FIG. 8 is an outer side elevation view of the garment shown in FIG. 6.

FIG. 9 is a top view of the garment shown in FIG. 6.

FIG. 10 is a bottom view of the garment shown in FIG. 6.

FIG. 11 is an inner side elevation view of a garment in accordance with some embodiments.

FIG. 12 is an inner side elevation view of a garment in accordance with some embodiments.

FIG. 13 is an outer side elevation view of a garment in accordance with some embodiments.

FIG. 14 is a front elevation view of the garment shown in FIG. 13.

FIG. 15 is a perspective view of the garment shown in FIG. 13.

In some of these figures, one or more portions of the foot, drawn using dashed lines, are shown to illustrate positions of various parts of garments, relative to the foot when such garments are worn. However, the foot is not part of the described garments.

### DETAILED DESCRIPTION OF PREFERRED EMBODIMENTS

As shown in the drawings, for purposes of illustration, preferred embodiments of posture improvement garments are shown and described. It will be appreciated that terms such as “front,” “back,” “top,” “bottom,” “anterior,” “posterior,” “superior,” “inferior,” “lateral,” “medial,” “side,” “short,” “long,” “up,” “down,” and “below” used herein are merely for ease of description and refer to the orientation of the components as shown in the figures. It should be understood that any orientation of the garments and the components thereof described herein is within the scope of the present invention.

It will also be understood that, although the terms first, second, etc. may be used herein to describe various elements, these elements should not be limited by these terms. These terms are only used to distinguish one element from another. For example, a first band could be termed a second band, and, similarly, a second band could be termed a first band, without departing from the scope of the present invention. The first band and the second band are both bands, but they are not the same band. In some embodiments, the first band and the second band have different properties (e.g., different elasticity values).

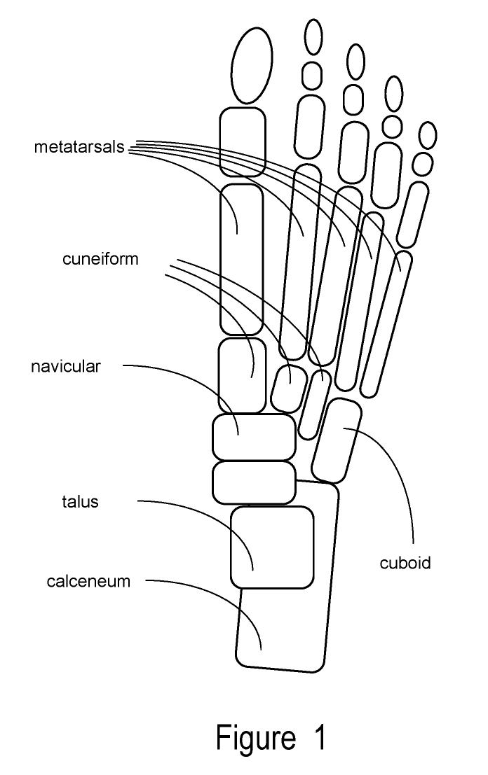

FIG. 1 is a schematic diagram illustrating a plan view of foot bones.

As explained above, a foot includes 26 bones. A forefoot portion (e.g., a front portion of the foot) includes toe bones (called phalanges) and five metatarsals (also called herein metatarsal bones). The metatarsal bones are located between tarsal bones and the phalanges, and are numbered the first, second, third, fourth, and fifth metatarsals from the medial side of the foot (e.g., the side of the hallux, which is also called a big toe). A midfoot portion (e.g., a middle portion of the foot) includes three cuneiform bones, a cuboid bone, and a navicular bone. The cuneiform bones are located between the navicular bone and metatarsal bones. The cuneiform bones are called, from the medial side of the foot, the first cuneiform (or the medial cuneiform), the second cuneiform (or the intermediate cuneiform or the middle cuneiform), and the third cuneiform (or the lateral cuneiform). A hindfoot portion, that corresponds to a heel and an ankle of the foot, includes a talus bone and a calcaneus bone (also called a heel bone). The talus bone provides support for leg bones (e.g., tibia and fibula).

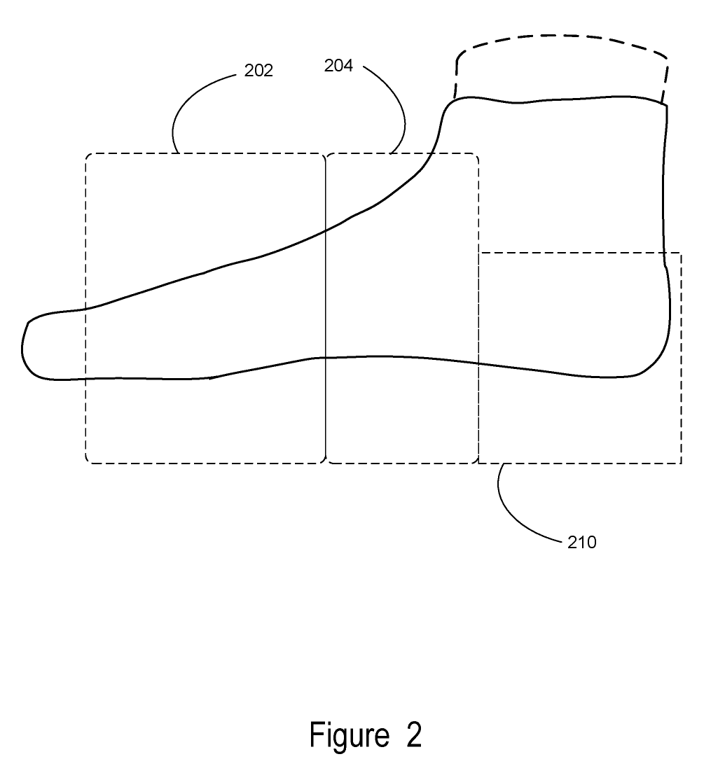

FIG. 2 is an inner side elevation view of a garment (e.g., a view from the medial side of the garment) in accordance with some embodiments.

The garment shown in FIG. 2 is a sock (when worn over a foot). The sock includes a foot portion. The foot portion corresponds to a portion of a foot between an ankle and a toe. In some embodiments, the foot portion includes an instep. As shown in FIG. 2, the foot portion includes an anterior portion 202 of the foot portion, and a posterior portion 204 of the foot portion. The sock also includes a heel portion 210. The heel portion 210 corresponds to a heel of the foot. In some embodiments, the sock is configured to cover the toe. In some embodiments, the sock is configured to expose the toe (e.g., one or more openings are provided in the anterior portion 202 of the foot portion to expose the toe).

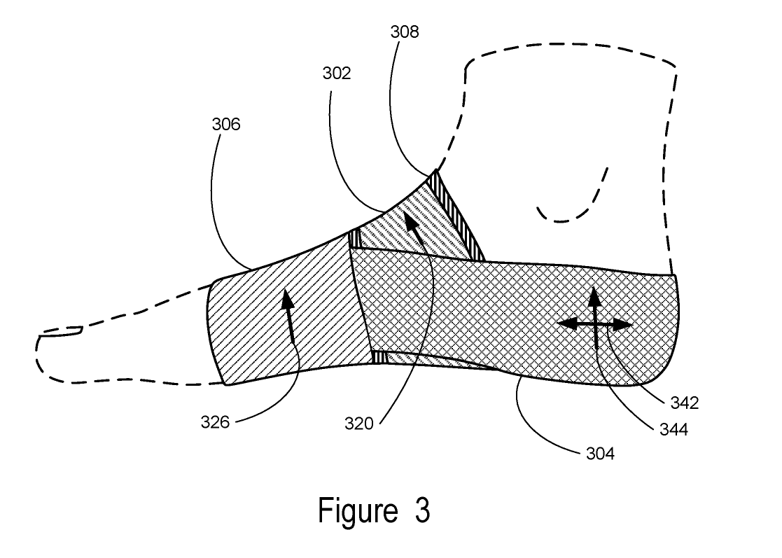

In some embodiments, the sock includes one or more bands described below with respect to FIGS. 3-15. In some embodiments, at least a subset of the one or more bands is located on an inner surface of the sock. In some embodiments, such bands located on the inner surface of the sock do not extend to an outer surface of the sock. In some embodiments, at least a subset of the one or more bands is located on an external surface of the sock. In some embodiments, such bands located on the outer surface of the sock do not extend to the inner surface of the sock. In some embodiments, at least a subset of the one or more bands is attached to the sock (e.g., by sewing, gluing, etc.). In some embodiments, at least a subset of the one or more bands is integrated with the sock (e.g., such bands are woven, concurrently with the sock, as part of the sock). In such embodiments, there is no need for separately sewing or attaching such bands to the sock. In some embodiments, when such bands are woven together as part of the sock, the sock is formed using a first thread and such bands are formed using a second thread that is distinct from the first thread. For example, the first thread has a first elasticity and the second thread has a second elasticity that is distinct from the first elasticity.

In some embodiments, one or more bands are formed by applying a first layer to an inner surface of the garment (e.g., on one or more portions of the inner surface of the garment corresponding respectively to the one or more bands described below with respect to FIGS. 3-15). In some embodiments, the first layer includes an elastic material (e.g., an elastomeric material, such as rubber). In some embodiments, the first layer is made of, or includes, an adherent material (e.g., a pressure-sensitive adhesive material, such as a polymer compound having a low hardness, which often provides tacky sensation upon contact with a skin). In some embodiments, the first layer includes an elastomeric material. In some cases, a mixture of a plasticizer and polyvinyl chloride particles, such as plastisol, is applied to the inner surface of the garment and cured (e.g., at about 180 degrees Celsius) to form the first layer. In some embodiments, a polysiloxane compound (e.g., silicone or a compound containing silicone) is applied to the inner surface of the garment to form the first layer. In some cases, the polysiloxane compound is cured (e.g., by heat and/or pressure or with one or more chemicals). In some embodiments, the first layer is configured to contact a skin of a wearer when the garment is worn on a foot of the wearer (e.g., the first layer is positioned on the inner surface of the garment). In some embodiments, the first layer increases friction between the garment and the wearer's skin. In some embodiments, the first layer facilitates adhesion of the garment to the wearer's skin. In some embodiments, in accordance with increased adhesion and/or friction, the first layer enhances the directional influence of the one or more bands of the garment (e.g., the first layer facilitates pulling one or more portions of the foot with the one or more bands of the garment).

In some embodiments, the garment includes one or more bands formed by applying a first layer to an inner surface of the garment and one or more bands that are not part of the first layer (e.g., one or more bands integrated with the sock, such as one or more woven bands, and/or one or more bands attached to the sock). For example, one or more bands are located on the inner surface of the garment to provide a direct contact and an increased friction and one or more bands are integrated with the sock or attached to the sock (e.g., on the outside of the sock) to provide a stronger pull.

In some embodiments, a garment includes a portion, less than all, of the sock described above with respect to FIG. 2 and also includes one or more bands described below with respect to FIGS. 3-15. In some embodiments, a garment includes one or more bands described below with respect to FIGS. 3-15 without a sock (e.g., the one or more bands described below are integrated to form a garment without a sock configured to envelope a foot).

FIG. 3 is an inner side elevation view (e.g., a view from the medial side of the garment) of a garment in accordance with some embodiments.

The garment includes a plurality of bands. In some embodiments, the garment includes one or more of: a band 302 that wraps around a posterior portion of a foot portion of the garment, a band 304 that wraps around a heel portion of the garment, and/or a band 306 that wraps around an anterior portion of the foot portion of the garment.

The band 302 facilitates pulling up a navicular bone of the foot (e.g., in the direction indicated by the arrow 320, which is included for illustrative purposes and not typically visible on a garment). The navicular bone is known as a keystone of arches of the foot. When someone has a flat foot, the navicular bone is positioned lower than its ideal position, thereby creating a flattened bottom of the foot. This lower position of the navicular bone may be caused by genetic factors and/or overall weakening of supporting muscles (e.g., lack of exercise of the supporting muscles). When the arch becomes flat, it loses the ability to effectively dampen impacts on the foot. By pulling up the navicular bone, the foot can better deal with impacts.

The band 304 provides support to a heel of the foot. The heel of the foot includes a fat pad, which absorbs impact. When a person stands and puts weight on the heel, the fat pad spreads and flattens to dissipate impact. However, when the person ages, the fat pad changes in shape (e.g., the fat pad gets thinner) and/or becomes less elastic (e.g., due to loss of hydration associated with aging). The band 304 provides support under and around the heel to better maintain the shape (and accordingly the function) of the fat pad, thereby improving the fat pad's ability to absorb and/or reduce shock.

In some embodiments, the band 306 provides support to at least a portion of the medial surface of the foot. In some embodiments, the band 306 has a superior portion and an inferior portion. In some embodiments, the medial portion of the band 306 is positioned to contact the medial surface of the foot. In some embodiments, the band 306 provides a pull on the medial portion of the band 306 toward the superior portion of the band 306 (e.g., in the direction indicated by arrow 326, which is included for illustrative purposes and not typically visible on a garment). In some embodiments, the band 306, when in use, is adjusted so that the band 306 provides a pull on the medial portion of the band 306 toward the superior portion of the band 306 (e.g., the medial portion of the band 306 is pulled up and toward the lateral portion of the band, while the garment is worn by a wearer, so that the band 306 provides a pull on the medial portion of the band 306 toward the superior portion of the band 306 while the garment is worn by the wearer).

In some embodiments, the band 306 provides support to at least a portion of the plantar surface of the foot. In some embodiments, the band 306 has a superior portion and an inferior portion. In some embodiments, the inferior portion of the band 306 is positioned to contact the plantar surface of the foot. In some embodiments, the band 306 provides a pull on the inferior portion of the band 306 toward the superior portion of the band 306, to support the plantar surface of the foot (e.g., in the direction indicated by arrow 326, which is included for illustrative purposes and not typically visible on a garment). In some embodiments, the band 306, when in use, is adjusted so that the band 306 provides a pull on the inferior portion of the band 306 toward the superior portion of the band 306 (e.g., the medial portion of the band 306 is pulled up and toward the lateral portion of the band, when the garment is worn by a wearer, so that the band 306 provides a pull on the inferior portion of the band 306 toward the superior portion of the band 306 while the garment is worn by the wearer).

In some embodiments, the garment also includes a friction layer 308. In some embodiments, the friction layer 308 is located underneath one or more of the bands. For example, in FIG. 3, the friction layer 308 is located underneath a medial portion of the band 302. The friction layer 308 further facilitates pulling up the navicular bone of the foot (e.g., in the direction indicated by the arrow 320). In some embodiments, the friction layer 308 has a higher rigidity than the band 302 (e.g., the friction layer 308 has a lower elasticity than the band 302). Thus, the friction layer 308 adds rigidity to the garment. For example, a portion of the garment that includes friction layer 308 is more rigid than portions of the garment that do not include friction layer 308. Also, for example, a portion of the garment that includes friction layer 308 is more rigid than if that portion did not include friction layer 308. In some cases, the increased rigidity is used to increase the support of the navicular bone. For example, the friction layer 308 is molded to have a particular shape, which enhances the support of the navicular bone when the garment is worn on the foot. In some embodiments, the friction layer 308, or the friction layer 308 in conjunction with the one or more of the bands above the friction layer 308, provide compression to the foot. In some embodiments, one or more of the bands 302, 304, and 306 have one or more friction layers. In some embodiments, each of the bands 302, 304, and 306 has a separate friction layer.

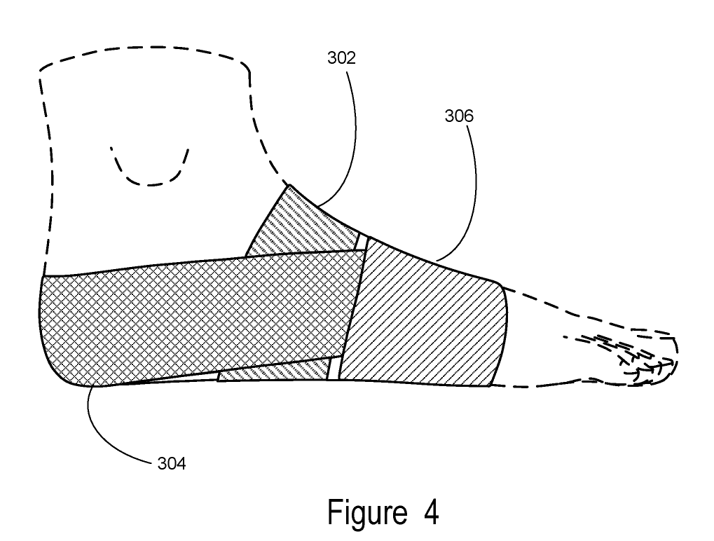

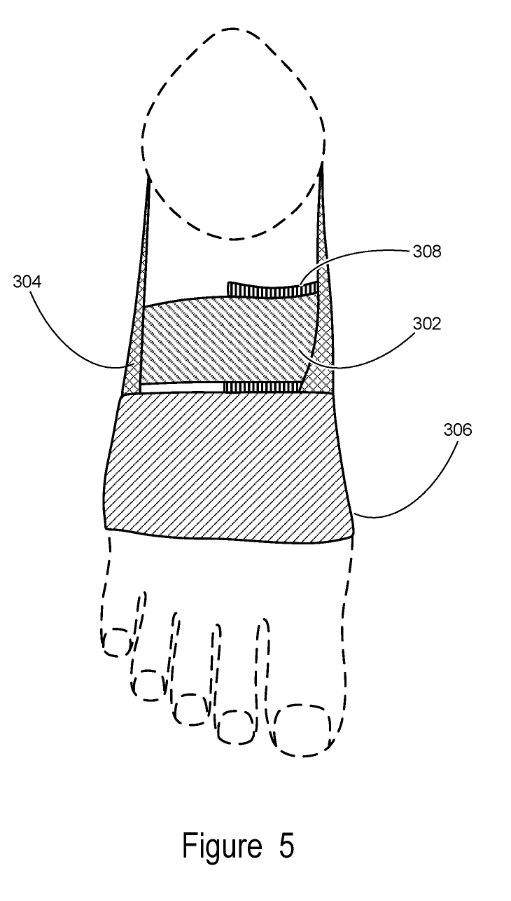

FIG. 4 is an outer side elevation view of the garment (e.g., a view from the lateral side of the garment) shown in FIG. 3, and FIG. 5 is a top view of the garment shown in FIG. 3. In some embodiments, the friction layer 308 is located on the medial portion of the band 302 (e.g., underneath the medial portion of the band 302, but not on the lateral portion (e.g., the outer side) of the band 302, as shown in FIG. 5. In FIG. 5, the friction layer 308 does not extend to the lateral portion of the band 302.

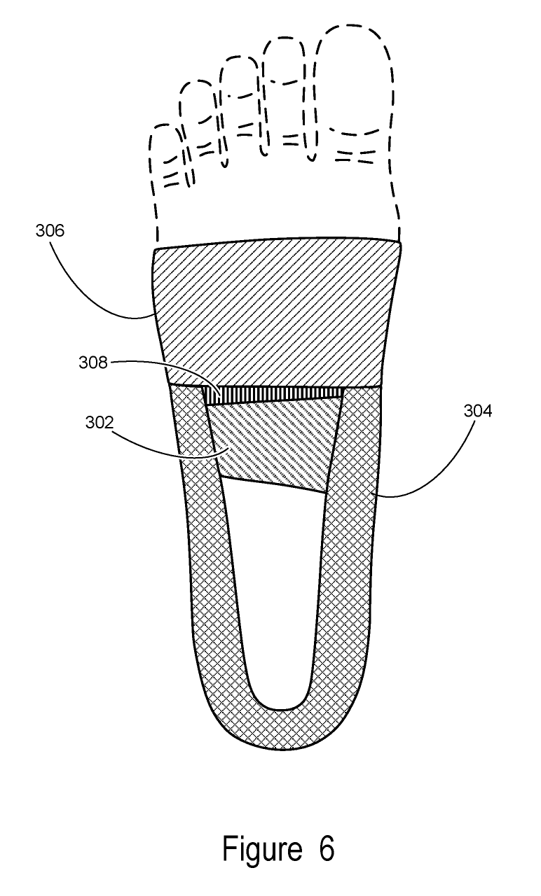

FIG. 6 is a bottom view of the garment shown in FIG. 3. The band 304 wraps around the heel portion of the garment. In some embodiments, the band 304 leaves a region of the heel portion uncovered by the band 304.

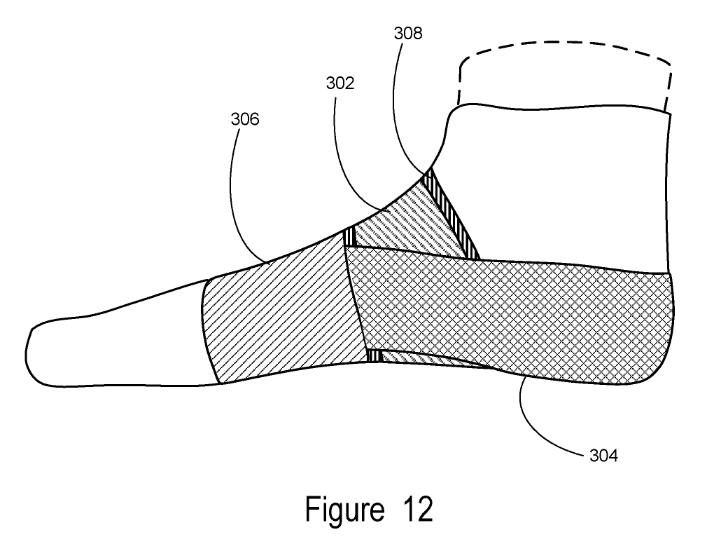

As explained above, FIGS. 3-6 illustrate a garment with a plurality of bands. In some embodiments, the garment consists of the plurality of bands shown in FIGS. 3-6 (or a subset thereof). For example, when the garment is worn on a foot, toes and a portion of a sole of the foot is exposed. In some other embodiments, the garment includes additional portions and/or features as shown in FIG. 12 (e.g., the plurality of bands is coupled with a sock).

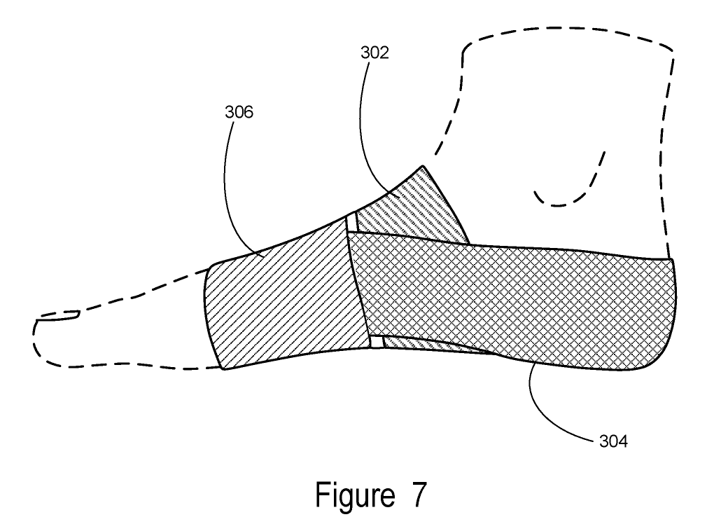

FIG. 7 is an inner side elevation view of a garment in accordance with some embodiments. FIG. 7 is similar to FIG. 3, except that the garment shown in FIG. 7 does not include the friction layer 308.

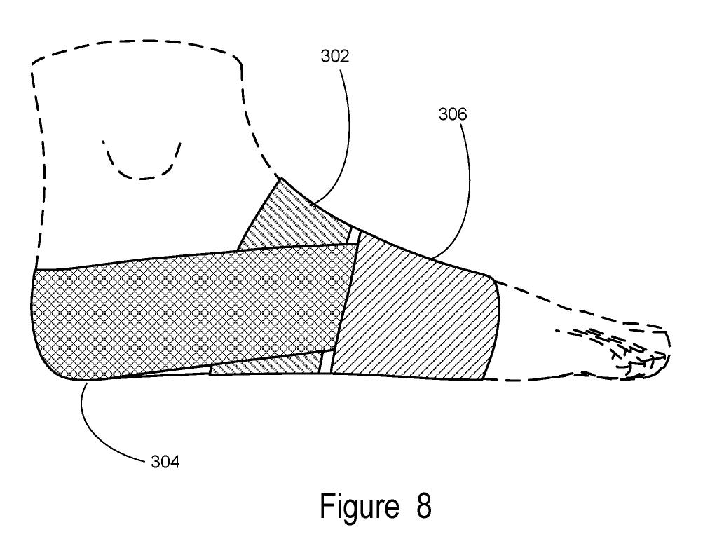

FIG. 8 is an outer side elevation view of the garment shown in FIG. 7. FIG. 8 is similar to FIG. 4.

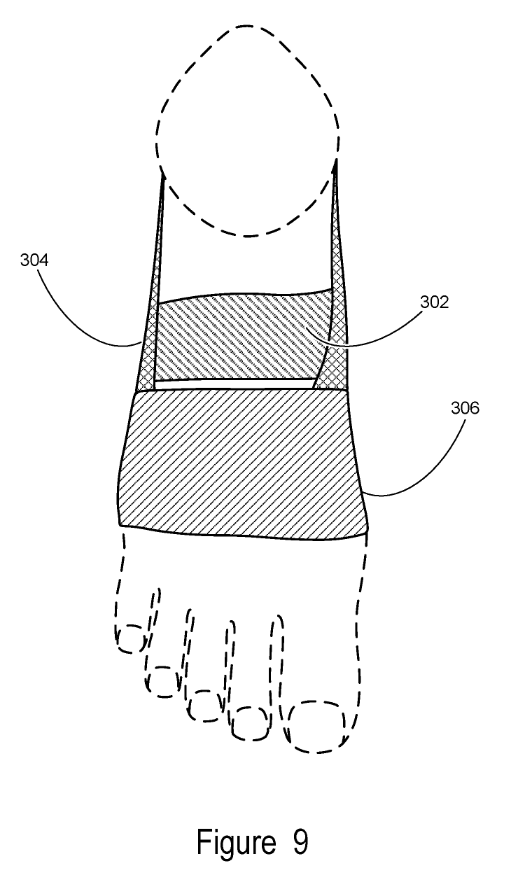

FIG. 9 is a top view of the garment shown in FIG. 7. FIG. 9 is similar to FIG. 5, except that the garment does not include the friction layer 308.

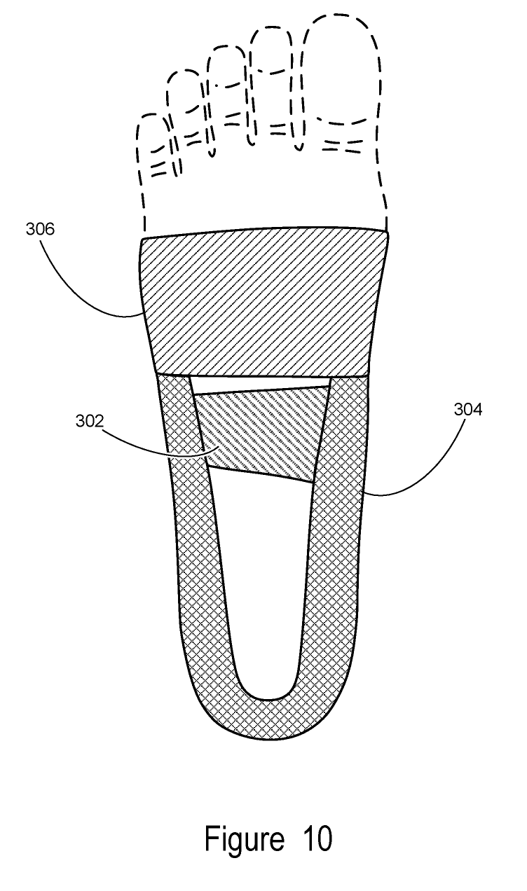

FIG. 10 is a bottom view of the garment shown in FIG. 7. FIG. 10 is similar to FIG. 6, except that the garment does not include the friction layer 308.

As explained above, FIGS. 7-10 illustrate a garment with a plurality of bands. In some embodiments, the garment consists of the plurality of bands shown in FIGS. 7-10 (or a subset thereof). For example, when the garment is worn on a foot, toes and a portion of a sole of the foot is exposed. In some other embodiments, the garment includes additional portions and/or features as shown in FIG. 12 (e.g., the plurality of bands is coupled with a sock).

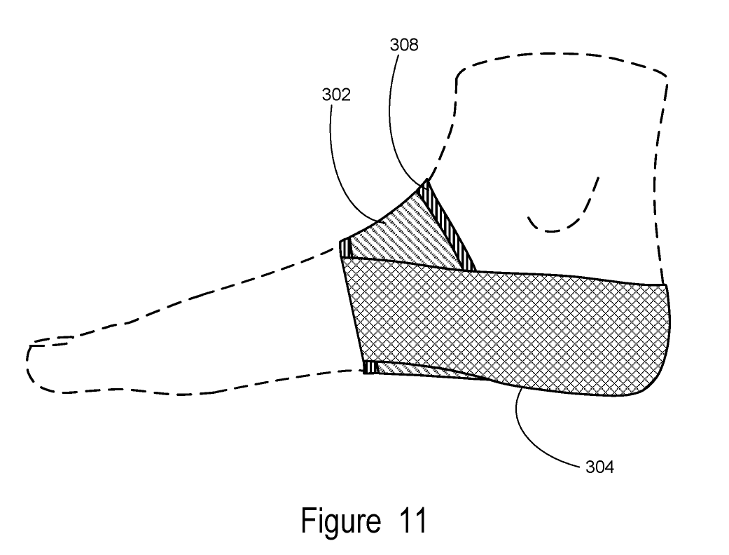

FIG. 11 is an inner side elevation view of a garment in accordance with some embodiments. FIG. 11 is similar to FIG. 3, except that the garment does not include the band 306. A person having ordinary skill in the art would understand the structure, configuration, and construction of the garment shown in FIG. 11 based on FIG. 11 and other drawings included herein, and their description. Thus, the outer side elevation view, the top view, and the bottom view of the garment shown in FIG. 11 are omitted for brevity.

In some embodiments, the garment consists of the plurality of bands shown in FIG. 11. For example, when the garment is worn on a foot, toes and a portion of a sole of the foot is exposed. In some other embodiments, the garment includes additional portions and/or features as shown in FIG. 12 (e.g., the plurality of bands is coupled with a sock).

FIG. 12 is an inner side elevation view of a garment in accordance with some embodiments. In FIG. 12, the plurality of bands shown in FIG. 3 is integrated with a sock. For example, the plurality of bands is integrated with the sock (e.g., by sewing, weaving, gluing, etc.). In some embodiments, the plurality of bands is located on the outside of the sock as shown in FIG. 12. In some embodiments, the plurality of bands is located on the inside of the sock. In some embodiments, a first subset of the plurality of bands is located on the outside of the sock and a second subset of the plurality of bands is located on the inside of the sock.

In some embodiments, the sock is an open toe sock. In some embodiments, the sock is a closed toe sock. In some embodiments, the sock extends to an ankle of the foot. In some embodiments, the sock does not extend to the ankle of the foot. In some embodiments, the sock extends above the ankle of the foot.

In some embodiments, the garment includes leggings. In some embodiments, the garment includes pants.

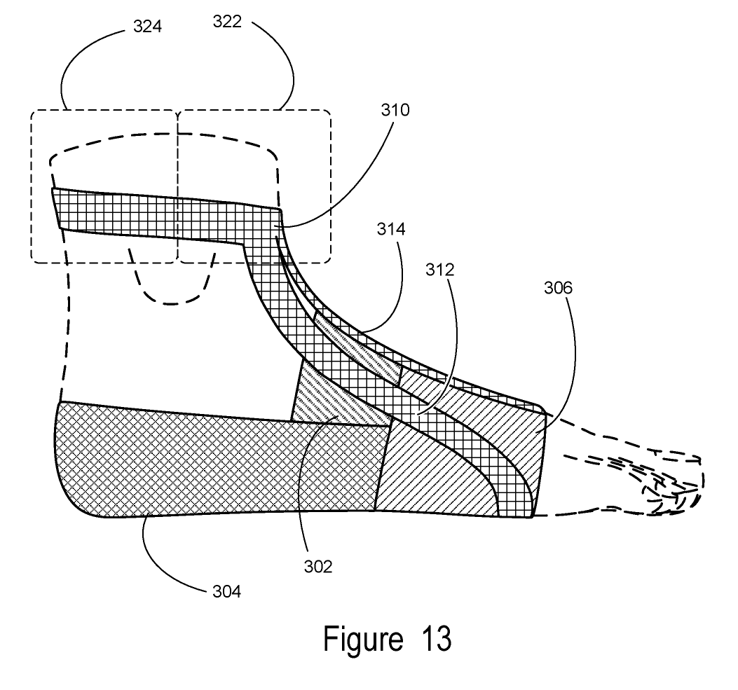

FIG. 13 is an outer side elevation view of a garment in accordance with some embodiments.

FIG. 13 is similar to FIG. 3, except that the garment includes an ankle band 310 and one or more lift bands (e.g., bands 312, 314, 316, and 318). In some embodiments, the ankle band 310 is configured (e.g., positioned) so that the ankle band 310 is located above a medial malleolus and a lateral malleolus of a foot. This prevents the ankle band from slipping down, and allows the ankle band to serve as an anchor for pulling other bands up (e.g., one or both of the bands 302 and 306).

FIG. 13 also illustrates that the ankle band 310 includes an anterior portion 322 (e.g., a portion of the ankle band 310 facing toes and/or the foot portion of the garment) and a posterior portion 324 (e.g., a portion of the ankle band 310 facing the heel side of the foot).

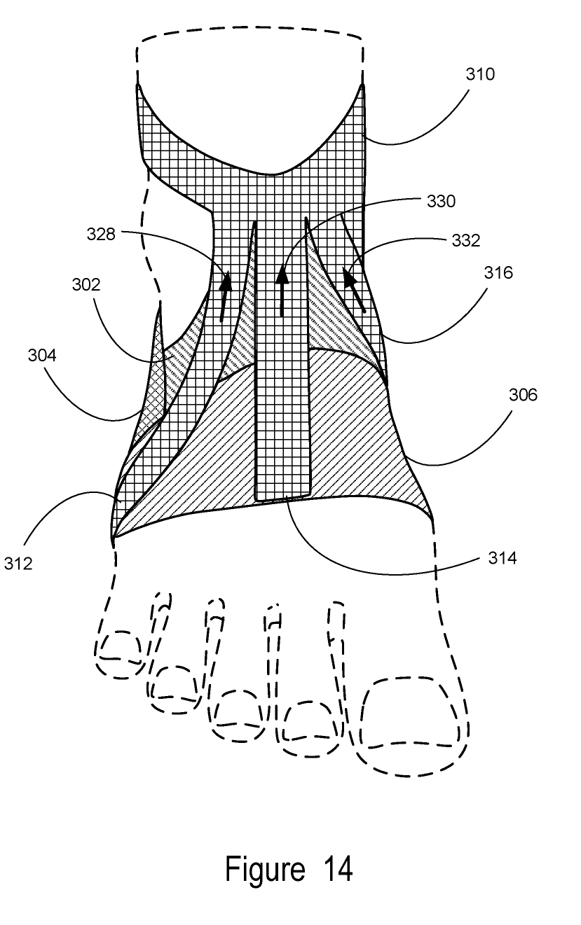

FIG. 14 is a front elevation view of the garment shown in FIG. 13.

As shown in FIG. 14, in some embodiments, the one or more lift bands (or a subset thereof) extend from an anterior portion of the ankle band 310 to a superior portion of the band 302 (e.g., the bands 312 and 314 extend from the anterior portion of the ankle band 310 to the superior portion of the band 302).

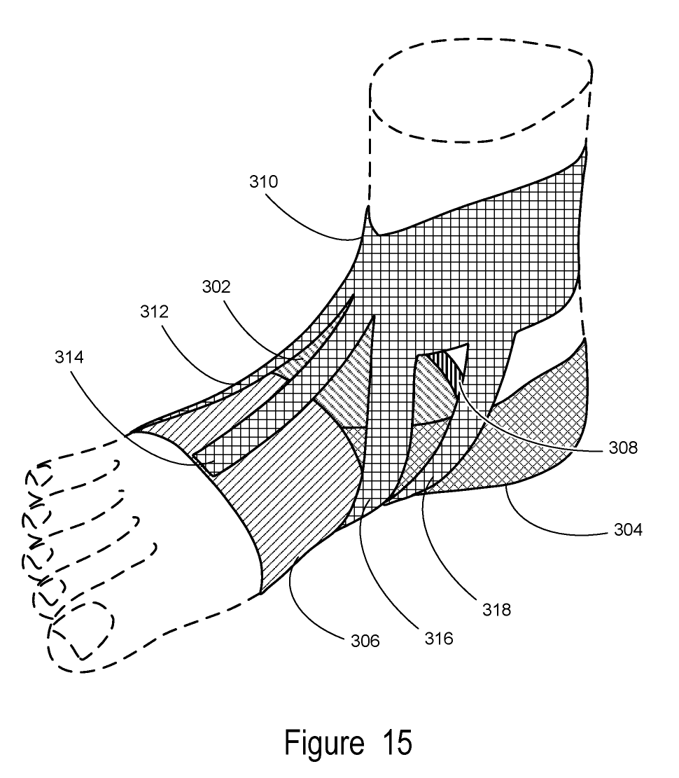

FIG. 15 is a perspective view of the garment shown in FIG. 13.

As shown in FIG. 15, in some embodiments, the one or more lift bands (or a subset thereof) extend from the anterior portion of the ankle band 310 to a medial portion of the band 302 (e.g., the band 316 extends from the anterior portion of the ankle band 310 to the medial portion of the band 302). This facilitates lifting the navicular bone of the foot, thereby reducing pain associated with pes planus (and/or plantar fasciitis).

In addition, in some embodiments, the one or more lift bands (or a subset thereof) extend from the ankle band 310 to a medial portion of the band 304 (e.g., the band 318 extends from the ankle band 310 to the medial portion of the band 304). This also facilitates lifting the navicular bone of the foot, thereby reducing pain associated with pes planus (and/or plantar fasciitis).

Certain embodiments of the garments may be best understood by reference to the above description taken in conjunction with the accompanying illustrative drawings.

In accordance with some embodiments, a garment includes a first band (e.g., band 302 in FIG. 3) configured to wrap around at least a portion of navicular, cuboid, and cuneiform bones of a wearer's foot when the garment is worn on the wearer's foot; and a second band (e.g., band 304 in FIG. 3) intersecting with the first band and configured to wrap around a heel of the wearer's foot when the garment is worn on the wearer's foot. The second band is configured to support medial and lateral surfaces and at least a portion of a plantar surface (e.g., an inferior surface) of the heel.

In some embodiments, the second band is configured to expose at least a portion of the plantar surface of the heel when the garment is worn on the wearer's foot (e.g., FIG. 6).

In some embodiments, the second band crisscrosses over at least a portion of navicular, cuboid, and cuneiform bones of the wearer's foot when the garment is worn on the wearer's foot.

In some embodiments, the second band is separate and distinct from the first band (e.g., as shown in FIG. 3, the band 304 is separate and distinct from the band 302).

In some embodiments, the second band is configured to pull the heel of the wearer's foot from an inferior surface of the wearer's foot toward a superior portion of the wearer's foot (e.g., in the direction indicated by the arrow 344, FIG. 3, which is included for illustrative purposes and not typically visible on a garment), and from a posterior portion of the wearer's foot toward an anterior portion of the wearer's foot (e.g., in the direction indicated by the arrows 342, FIG. 3, which is also included for illustrative purposes and not typically visible on a garment) when the garment is worn on the wearer's foot. For example, the band 304 pulls up and pulls forward (e.g., toward the toes) the heel of the wearer's foot, thereby maintaining the shape and position of the fat pad and reducing spreading of the fat pad when there is an impact on the heel (e.g., as indicated by the arrows 342, FIG. 3, the band 304 pulls the heel of the wearer's foot toward the toes, and vice versa).

In some embodiments, the garment includes a third band (e.g., band 306 in FIG. 3) configured to wrap around at least a portion of metatarsals of the wearer's foot when the garment is worn on the wearer's foot.

In some embodiments, the second band extends from a medial portion of the third band to a lateral portion of the third band while wrapping around the heel of the wearer's foot when the garment is worn on the wearer's foot. This allows the second band to use the third band as an anchor (or an additional anchor) when pulling the heel of the wearer's foot forward.

In some embodiments, the garment includes a plurality of bands configured to pull the third band toward an ankle (e.g., in a neutral position) of a wearer when the garment is worn on the wearer's foot. For example, bands 312, 314, and 316 in FIG. 14 pull the band 306 toward the ankle of the wearer (or toward the ankle band 310) (e.g., in the directions indicated by the arrows 328, 330, and 332, FIG. 14, which are included for illustrative purposes and not typically visible on a garment).

In some embodiments, the garment includes an ankle band (e.g., the ankle band 310 shown in FIG. 13) configured to wrap around the ankle of the wearer. The plurality of bands extends from the third band to the ankle band. For example, in FIG. 14, the bands 312, 314, and 316 extend from the band 306 to the ankle band 310. This facilitates maintaining and/or improving the arches of the foot (e.g., by lifting the navicular bone of the foot).

In some embodiments, the ankle band is separate and distinct from the second band (e.g., in FIG. 13, the ankle band 310 is separate and distinct from the band 304). In some embodiments, the ankle band is separate and distinct from the first band (e.g., in FIG. 13, the ankle band 310 is separate and distinct from the band 302). Alternatively, in some embodiments, the ankle band is integrated with the first band.

In accordance with some embodiments, a garment includes an ankle band that includes an anterior portion and a posterior portion. For example, in FIG. 13, the ankle band 310 includes the anterior portion 322 and the posterior portion 324. In some embodiments, the ankle band is positioned to wrap around an ankle of a wearer above a medial malleolus and a lateral malleolus of the wearer when the garment is worn on a foot of the wearer. In some embodiments, the anterior portion corresponds to a portion (e.g., one half) of the ankle band that is located toward the foot portion, and the posterior portion corresponds to a portion (e.g., one half) of the ankle band that is located away from the foot portion. The garment also includes a foot portion that includes a first band (e.g., the band 302 in FIG. 13) wrapping around the foot portion. The first band has a superior portion and an inferior portion. The garment further includes a plurality of lift bands extending from the anterior portion of the ankle band to at least the superior portion of the first band so as to provide a pull on the superior portion of the first band toward the anterior portion of the ankle band. For example, the bands 312, 314, and 316 in FIG. 14 pull the superior portion of the band 302 toward the anterior portion of the ankle band 310 (e.g., in the directions indicated by the arrows 328, 330, and 332, FIG. 14).

In some embodiments, one or more lift bands of the plurality of lift bands extend from the anterior portion of the ankle band to an inferior portion of the first band. In some embodiments, the bands 316 and 318 extend beneath the band 302 as shown in FIG. 15.

In some embodiments, the foot portion includes an anterior portion and a posterior portion (e.g., the anterior portion 202 and the posterior portion 204 in FIG. 2). The first band is located at least partially within the posterior portion of the foot portion (e.g., the first band 302 is located at least partially within the posterior portion of the foot portion). In some embodiments, the first band is located adjacent to the ankle band. In some embodiments, the first band is located within one inch from the ankle band (e.g., a minimum distance between the first band and the ankle band is less than one inch, or the first band touches or overlaps with at least a portion of the ankle band). In some embodiments, the first band is positioned to wrap around at least a portion of navicular, cuboid, and cuneiform bones of a wearer's foot when the garment is worn on the wearer's foot. In some embodiments, a distance (e.g., a minimum distance or a shortest distance) between the first band and the ankle band is less than a distance (e.g., a minimum distance or a shortest distance) between a second band around the foot portion and the ankle band (e.g., the first band is located closer to the ankle band than the second band is to the ankle band).

In some embodiments, the garment includes a second band (e.g., the band 306 in FIG. 3) that wraps around the foot portion and is located at least partially within the anterior portion of the foot portion. The second band has a superior portion and an inferior portion, and, in some embodiments, one or more lift bands of the plurality of lift bands extend from the anterior portion of the ankle band to at least the superior portion of the second band so as to provide a pull on the superior portion of the second band toward the anterior portion of the ankle band. For example, in FIG. 15, the bands 312, 314, and 316 extend from the anterior portion of the ankle band 310 to the superior portion of the band 306 and pull the superior portion of the band 306 toward the anterior portion of the ankle band 310) (e.g., in the directions indicated by the arrows 328, 330, and 332, FIG. 14). In some embodiments, the second band is located parallel to the first band. In some embodiments, the second band is located more than one inch away from the ankle band (e.g., a minimum distance between the second band and the ankle band is greater than one inch). In some embodiments, one or more lift bands of the plurality of lift bands extend from the anterior portion of the ankle band to an inferior portion of the second band. In some embodiments, the second band is positioned to wrap around at least a portion of metatarsals of a wearer's foot when the garment is worn on the wearer's foot.

In some embodiments, the foot portion includes an anterior portion and a posterior portion; and the first band is located at least partially within the anterior portion of the foot portion. In some embodiments, the second band is located by more than one inch away from the ankle band. In some embodiments, at least one or more lift bands of the plurality of lift bands extend from the anterior portion of the ankle band to an inferior portion of the second band. In some embodiments, the first band is positioned to wrap around at least a portion of metatarsals of a wearer's foot when the garment is worn on the wearer's foot.

In some embodiments, the first band has a medial portion (e.g., toward an inside of the foot, closer to the midline of the wearer's body) and a lateral portion (e.g., toward an outside of the foot, further away from the midline of the wearer's body). The first band is positioned to contact a skin of a wearer when the garment is worn on a foot of the wearer. In some embodiments, the garment also includes a friction layer located on the medial portion of the first band to pull up a navicular bone of the wearer when the garment is worn on the foot of the wearer (e.g., the friction layer 308 in FIG. 15). In some embodiments, the friction layer is integrated with the first band. In some embodiments, the friction layer is distinct and separate from the first band.

Generally, the navicular bone is lifted and supported naturally by a healthy tone of the posterior tibialis muscle. Based on the placement, skin contact, and directional influence of the band (e.g., the first band and/or the friction layer) contacting cutaneous nerve endings in the skin, the posterior tibialis muscle is cued to switch on and provide the lift and support of the navicular bone (which is often known as the keystone bone of the arches of the foot). For example, the placement and direction of the first band facilitates the necessary muscle tone to improve the alignment and function (mobility, stability, and strength) of a medial longitudinal arch of the foot. In some embodiments, this effect is enhanced by other features described herein (e.g., the friction layer and/or other bands).

In some embodiments, the first band has a medial portion and a lateral portion. The first band has a first thickness at the medial portion and a second thickness, that is less than the first thickness, at the lateral portion. For example, the first band is thicker toward the inside of the foot than toward the outside of the foot. This provides additional support for pulling the navicular bone up.

In some embodiments, the garment includes a heel portion; and a heel band (e.g., the band 304 in FIG. 15) intersecting with the first band and wrapping around at least a portion of the heel portion so as to pull the heel portion toward the foot portion. In some embodiments, the heel band also provides lateral support on the heel portion (e.g., support for the sides of the heel) when the garment is worn on the wearer's foot. In some embodiments, the first band has a first elasticity and the heel band has a second elasticity that is distinct from the first elasticity (e.g., the heel band is more elastic than the first band).

In some embodiments, the plurality of lift bands includes at least one lift band extending from the anterior portion of the ankle band to at least the superior portion of the first band at least partially over a middle metatarsal bone (e.g., the band 314 in FIG. 14).

In some embodiments, the first band has a medial portion and a lateral portion. At least two lift bands of the plurality of lift bands extend from the anterior portion of the ankle band to the medial portion of the first band (e.g., the bands 316 and 318 in FIG. 15). At least one lift band of the plurality of lift bands extends from the anterior portion of the ankle band to the lateral portion of the first band (e.g., the bands 312 and 314 in FIG. 14).

In some embodiments, the garment is a sock. In some embodiments, the garment is an open-toe sock. In some embodiments, the garment is a closed-toe sock. In some embodiments, the garment is a toe sock. In some embodiments, the garment is a slip-on brace. In some embodiments, the garment does not have a heel portion. In some embodiments, the garment has a leg portion (e.g., pants). In some embodiments, the garment does not have a leg portion.

In accordance with some embodiments, a garment includes a foot portion that includes a first band wrapping around the foot portion (e.g., the band 302 in FIG. 11). The first band has a medial portion and a lateral portion. The first band is positioned to contact a skin of a wearer when the garment is worn on a foot of the wearer. In some embodiments, the garment also includes a friction layer (e.g., the friction layer 308 in FIG. 11) located on the medial portion of the first band to pull up (and/or support) a navicular bone of the wearer when the garment is worn on the foot of the wearer.

In accordance with some embodiments, a method for reducing pain associated with pes planus includes wearing, on a foot, a garment that includes an ankle band that includes an anterior portion and a posterior portion; a foot portion that includes a first band (e.g., the band 302 in FIG. 14) wrapping around the foot portion; and a plurality of lift bands (e.g., the bands 312, 314, and 316 in FIG. 14) extending from the anterior portion of the ankle band to at least the superior portion of the first band. The first band has a superior portion and an inferior portion. The method also includes pulling the superior portion of the first band toward the anterior portion of the ankle band and lifting a navicular bone on a medial portion of the foot. In some embodiments, this method reduces pain associated with ples planus (and/or plantar fasciitis) by improving the function of the foot.

It should be noted that characteristics of garments described herein with respect to FIGS. 2-15 are also applicable in an analogous manner to the method described above in the preceding paragraph. For example, the garment may include a second band (e.g., the band 304) and/or a third band (e.g., the band 306) described herein with reference to FIGS. 3-6. For brevity, these details are not repeated here.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. For example, the garment may not include one or more features described herein (e.g., a garment may omit any of the band 302, the band 304, and/or the band 306).

The embodiments were chosen and described in order to best explain the principles of the invention and its practical applications, to thereby enable others skilled in the art to best use the invention and various described embodiments with various modifications as are suited to the particular use contemplated.

Accordingly, although exemplary embodiments of the invention have been shown and described, it is to be understood that all the terms used herein are descriptive rather than limiting, and that many changes, modifications, and substitutions may be made by one having ordinary skill in the art without departing from the spirit and scope of the invention.

What is claimed is:

1. A garment, comprising:
a first band configured to wrap around at least a portion of navicular, cuboid, and cuneiform bones of a wearer's foot when the garment is worn on the wearer's foot; and
a second band intersecting with the first band and configured to wrap around a heel of the wearer's foot when the garment is worn on the wearer's foot, wherein the second band is configured to support medial and lateral surfaces and at least a portion of a plantar surface of the heel.

2. The garment of claim 1, wherein:
the second band is configured to expose at least a portion of the plantar surface of the heel when the garment is worn on the wearer's foot.

3. The garment of claim 1, wherein:
the second band crisscrosses over at least a portion of navicular, cuboid, and cuneiform bones of the wearer's foot when the garment is worn on the wearer's foot.

4. The garment of claim 1, wherein:
the second band is separate and distinct from the first band.

5. The garment of claim 1, wherein:
the second band is configured to pull the heel of the wearer's foot from an inferior surface of the wearer's foot toward a superior portion of the wearer's foot, and from a posterior portion of the wearer's foot toward an anterior portion of the wearer's foot when the garment is worn on the wearer's foot.

6. The garment of claim 1, further comprising:
a third band configured to wrap around at least a portion of metatarsals of the wearer's foot when the garment is worn on the wearer's foot.

7. The garment of claim 6, wherein:
the second band extends from a medial portion of the third band to a lateral portion of the third band while wrapping around the heel of the wearer's foot when the garment is worn on the wearer's foot.

8. The garment of claim 6, further comprising:
a plurality of bands configured to pull the third band toward an ankle of a wearer when the garment is worn on the wearer's foot.

9. The garment of claim 8, further comprising:
an ankle band configured to wrap around the ankle of the wearer, wherein the plurality of bands extends from the third band to the ankle band.

10. The garment of claim 9, wherein:
the ankle band is separate and distinct from the second band.

11. A garment, comprising:
an ankle band that includes an anterior portion and a posterior portion;
a foot portion that includes a first band wrapping around the foot portion, wherein the first band has a superior portion and an inferior portion; and
a plurality of lift bands extending from the anterior portion of the ankle band to at least the superior portion of the first band so as to provide a pull on the superior portion of the first band toward the anterior portion of the ankle band.

12. The garment of claim 11, wherein:
the foot portion includes an anterior portion and a posterior portion; and
the first band is located at least partially within the posterior portion of the foot portion.

13. The garment of claim 12, further comprising:
a second band that wraps around the foot portion and is located at least partially within the anterior portion of the foot portion, wherein the second band has a superior portion and an inferior portion, and one or more lift bands of the plurality of lift bands extend from the anterior portion of the ankle band to at least the superior portion of the second band so as to provide a pull on the superior portion of the second band toward the anterior portion of the ankle band.

14. The garment of claim 11, wherein:
the foot portion includes an anterior portion and a posterior portion; and
the first band is located at least partially within the anterior portion of the foot portion.

15. The garment of claim 11, wherein:
the first band has a medial portion and a lateral portion;
the first band is positioned to contact a skin of a wearer when the garment is worn on a foot of the wearer; and
the garment also includes a friction layer located on the medial portion of the first band to pull up a navicular bone of the wearer when the garment is worn on the foot of the wearer.

16. The garment of claim 11, wherein:
the first band has a medial portion and a lateral portion; and
the first band has a first thickness at the medial portion and a second thickness, that is less than the first thickness, at the lateral portion.

17. The garment of claim 11, further comprising:
a heel portion; and
a heel band intersecting with the first band and wrapping around at least a portion of the heel portion so as to pull the heel portion toward the foot portion.

18. The garment of claim 11, wherein the plurality of lift bands includes at least one lift band extending from the anterior portion of the ankle band to at least the superior portion of the first band at least partially over a middle metatarsal bone.

19. The garment of claim 18, wherein:
the first band has a medial portion and a lateral portion;
at least two lift bands of the plurality of lift bands extend from the anterior portion of the ankle band to the medial portion of the first band; and
at least one lift band of the plurality of lift bands extends from the anterior portion of the ankle band to the lateral portion of the first band.

20. A garment, comprising:
a foot portion that includes a first band wrapping around the foot portion, wherein the first band has a medial portion and a lateral portion;
the first band is positioned to contact a skin of a wearer when the garment is worn on a foot of the wearer; and
the garment also includes a friction layer located on the medial portion of the first band to pull up a navicular bone of the wearer when the garment is worn on the foot of the wearer.
